(19) United States
(12) Plant Patent Application Publication
(10) Pub. No.: US 2003/0192101 P1
(43) Pub. Date: Oct. 9, 2003

(54) Chrysanthemum plant named 'Chasky'

(76) Inventor: Jean-Pierre Challet, Nuaille (FR)

Correspondence Address: BURNS, DOANE, SWECKER & MATHIS, L.L.P., Benton S. Duffett, Jr., P.O. Box 1404, Alexandria, VA, 22313-1404, US

(21) Appl. No.: 10/117,094

(22) Filed: Apr. 8, 2002

(51) Int. Cl. A01H 5/00

(52) U.S. Cl. PLT/294

(57) ABSTRACT

A new and distinct Chrysanthemum cultivar is provided that is the result of a controlled breeding program. Attractive anemone-type blossoms having plain white ray florets are formed in profusion. The central cushion is well developed and bears disc florets that progressively change with maturity from green to yellow to creamy white. The growth habit is low, very compact and well-branched and yields a generally uniform and spherical overall plant. The foliage is small and deep green and contrasts well with the yellow blossom coloration. The response time is approximately seven and one-half weeks. The plant grows well in pots, and the blossoms commonly last approximately three to four weeks.

### BOTANICAL/COMMERCIAL CLASSIFICATION

Dendranthema grandifloria/Decorative Pot Mum

### VARIETAL DENOMINATION cv. ‘Chasky’

### SUMMARY OF THE INVENTION

The present invention comprises a new and distinct cultivar of Chrysanthemum, botanically known as Dendranthema grandiflora, and hereafter is referred to by the cultivar name ‘Chasky’.

The new cultivar of the present invention was created at Nuaillé, France during the course of a controlled breeding program. The seeds resulting from the cross were sown and small plants were obtained which were physically and biologically different from each other. Selective study resulted in the identification of a single plant of the new variety of the present invention.

It was found that the new Chrysanthemum cultivar of the present invention displays:

(a) a low very compact, well-branched, and generally spherical growth habit,

(b) forms attractive long-lasting decorative white anemone-type blossoms,

(c) forms attractive deep green leaves that are small in size, and

(d) an ability to grow well in pots.

The new cultivar can be grown singly or in clumps in pots. It also can be grown in the landscape. The white blossoms contrast nicely with the deep green foliage. The blossoms have been found to be long lasting and commonly last approximately three to four weeks on the plant. The plant is self-branching. Pinching is helpful to further enhance branching and produces a large number of shoots; however, such pinching is not necessary since the plant already is inherently well branched.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chasky’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPH

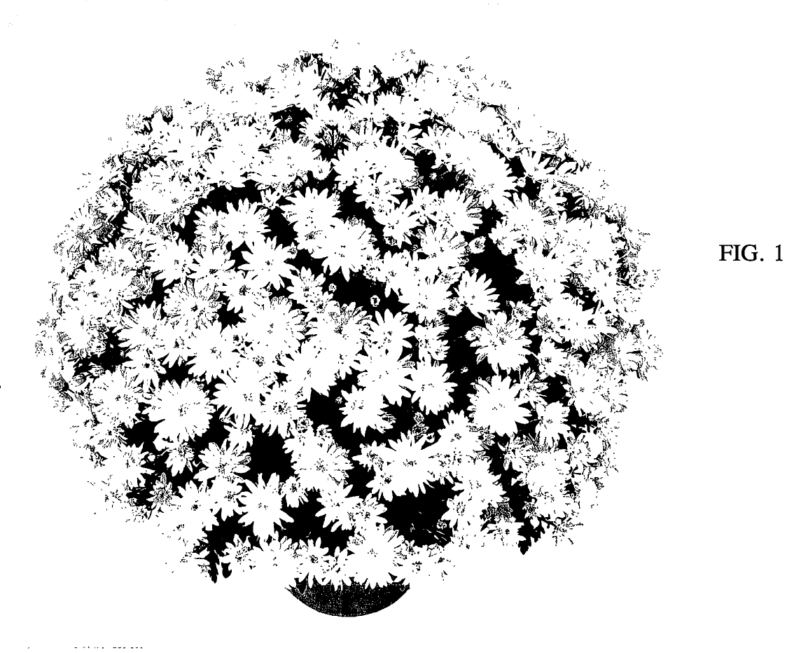

The accompanying FIG. 1 depicts a typical plant of ‘Chasky’ cultivar while growing in a pot in a greenhouse at Nuaillé, France. The attractive very compact spherical growth habit, attractive white anemonetype blossoms, and deep green foliage are illustrated.

### DETAILED DESCRIPTION

The plants described were grown in pots in a greenhouse at Nuaillé, France. No growth regulation was used. The growing conditions approximate those commonly utilized for the commercial production of decorative pot mums.

General Appearance:

Short and stocky, very compact, with a generally spherical growth habit.

Foliage:

Attractive deep green.

Flowers:

The ray florets of the anemone-type blossoms are petal-shaped and plain white in coloration. The central cushion is well developed and bears disc florets that progressively change with maturity from green to yellow to creamy white.

Flower Duration:

Very long lasting with the blossoms commonly lasting approximately three to four weeks on the plant.

Response Time:

The time between the beginning of the short day period and the flowering date at normal temperature and light intensity is approximately seven and one-half weeks.

Usage:

Decorative pot mum.

I claim:

1. A new and distinct cultivar of Chrysanthemum plant that displays:
(a) a low very compact, well-branched, and generally spherical growth habit,
(b) forms attractive long-lasting decorative white anemone-type blossoms,
(c) forms attractive deep green leaves that are small in size, and
(d) an ability to grow well in pots;
substantially as illustrated and described.
